(19) United States
(12) Patent Application Publication
(10) Pub. No.: US 2025/0297298 A1
(43) Pub. Date: Sep. 25, 2025

(54) FERMENTATION PROCESS INTENDED TO PRODUCE METABOLITES WITH EPIGENETIC PROPERTIES USING BACTERIA EVOLVED IN THE EXTREME CONDITIONS OF SPACE

(71) Applicant: Innovation Labo Sciences, Co. Ltd, Tokyo (JP)

(72) Inventors: Mizuho Nasu, Tokyo (JP); Bejit Ideas, Tokyo (JP); Marshall Porterfield, Lafayette, IN (US); Vera Mulyani, Santa Monica, CA (US); Jennifer Fogarty, Seabrook, TX (US)

(21) Appl. No.: 18/951,317

(22) Filed: Nov. 18, 2024

(30) Foreign Application Priority Data
Nov. 17, 2023 (FR) 2312657

(51) Int. Cl. C12P 39/00; A61K 45/06; C12R 1/01

(52) U.S. Cl. CPC C12P 39/00; A61K 45/06; C12R 2001/01

(57) ABSTRACT

Fermentation process intended to produce metabolites modulating the epigenetic imprint characterized in that the fermentation is carried out with biosourced ingredients at a temperature between 45 and 50° C. for 180 hours by a co-culture of at least three bacterial strains that have been evolved by exposure to space environment conditions.

### FIELD OF THE INVENTION

The present invention relates to a fermentation process intended to produce metabolites having epigenetic imprint modulation properties, characterized in that the fermentation is carried out with bio-sourced ingredients at a temperature between 45 and 50° C. for 180 hours by a co-culture of at least three bacterial strains that have evolved under stress, by exposure to space environment conditions.

### BACKGROUND OF THE INVENTION

Epigenetics is defined as the interaction of genes with their environment which conditions the phenotype of the organism, that is to say all of its characteristics.

We know that fermentation is caused by microorganisms such as bacteria, yeasts or molds. The latter are living forms invisible to the naked eye that multiply very quickly and are found in our environment in large quantities. Certain organic materials can ferment thanks to ferments that will transform sugars and proteins into alcohol, acid and carbon dioxide. These will then modify the environment and prevent the multiplication of undesirable microorganisms, which allows in particular the preservation of fermented products. This process is most often done in the absence of oxygen. Fermentation has been used empirically for thousands of years; in particular during the manufacture of bread, alcoholic beverages (such as wine and beer for example) and vinegar. The bacterial agents responsible for fermentation were discovered in the 17th century and its industrial applications developed later, in the 20th century. In 1677, Antonie van Leeuwenhoek invented the microscope, which allowed the first scientific work on yeasts to see the light of day. In 1836, Cagniard-La Tour, Schwann and Kützing published microscopic observations that led to the conclusion that yeast was a living organism that reproduced by budding. The different fermentations were studied by Louis Pasteur from 1857. He then demonstrated by isolating and cultivating bacteria or yeasts responsible for fermentation that it was a chemical but also biological process.

Patent application US202217718520 discloses a nutritional composition containing metabolites of bacteria obtained by fermentation of the microbiota. Patent application CN114403400 (A) discloses a method for controlling and optimizing the production of bacterial metabolites using fermentation. This patent explains the fermentation steps that are used to generate numerous metabolites with beneficial bacterial flora and which allow industrialization of the process. However, the problem with this process is that the fermentation time is very long since it is a long fermentation lasting 5 and 10 years. Patent application CN107488624 (A) discloses a rapid cultivation method that adopts a modern fermentation process using mycelium to obtain many metabolites. The advantages of this method are the saving of resources and the short cultivation time. Patent application FR3062396 discloses a fermentation process intended to produce metabolites with anti-inflammatory properties and increased mitochondrial activity, characterized in that the bacteria used for the cultivation are extracted from the microbiota of healthy centenarians. The process takes place in several stages for a total duration of at least 120 hours, including prior sterilization of the fermented ingredients, inoculation of the bacteria and cultivation of the mixture at a temperature between 30° C. and 45° C. The fermented ingredients are of bio-sourced origin only and are obtained without the use of pesticides. Patent application FR3054441 discloses a dermocosmetic composition intended to be used topically to restore the balance of the skin and intestinal microbiota, characterized in that it contains at least 150 different metabolites originating from the slow fermentation of a mixture of several plants by a combination of lactobacteria containing at least one strain of bacteria originating from the thousand-year-old Olea europaea plant and particularly its leaves. None of these patents demonstrate an effect on the epigenetic imprint.

It was by studying the genome and metabolic activity of bacteria exposed to extreme conditions that the applicant discovered that bacteria exposed to space conditions could produce metabolites with the capacity to modulate the epigenetic imprint. The applicant therefore developed a fermentation process using evolved bacteria.

The applicant describes below the properties of this fermentation process based on bio-sourced ingredients and a co-culture of at least 3 bacterial strains. Its use allows the production of metabolites modulating the epigenetic imprint by modulating the enzymes regulating epigenetics and increasing the telomerase rate.

### SUMMARY OF THE INVENTION

The subject of the present invention therefore relates to a fermentation process intended to produce metabolites having epigenetic imprint modulation properties, characterized in that the fermentation is carried out with bio-sourced ingredients at a temperature between 45 and 50° C. for 180 hours by a co-culture of at least 3 bacterial strains that have been evolved by exposure to space conditions. The evolution, or modification of said at least three bacteria takes place in particular by exposure to UV radiation, this leading to genetic and/or epigenetic modifications, in particular epigenetic.

It should be noted that the fermentation process is characterized in that the bacteria used for fermentation are taken from the following list: Lactobacillus fermentum, Lactobacillus casei, Lactobacillus acidophilus, Bifidobacterium longum, Bifidobacterium bifidum, Bifidobacterium adolescentis, Bifidobacterium infantis, Lactobacillus acidophilus, Lactobacillus brevis, Lactobacillus Jensenii, Lactobacillus paracasei subsp. Paracasei, Lactobacillus gasseri, Lactobacillus reuteri, Lactobacillus delbrueckii subsp. Bulgaricus, Lactobacillus helveticus, Lactococcus lactis, Lactobacillus casei subsp. Casei, Lactobacillus rhamnosus, Lactobacillus delbrueckii subsp. Delbrueckii, Enterococcus faecium and Streptococcus thermophilus, Akkermansia muciniphila.

In one embodiment, the bacterial strains were evolved by exposure to space conditions consisting of exposure to UV-B type UV radiation at a dose of 0.1 to 3.0 W/m2.

Advantageously, the fermentation process is characterized in that the fermented ingredients are of bio-sourced origin only, which are obtained without the use of pesticides and which are previously sterilized.

According to a particular embodiment, the bio-sourced ingredients are chosen from soybeans, olive leaves and a mixture of these.

The invention also relates to a method for the preparation of a pharmaceutical or cosmetic composition comprising:
(i) a fermentation step according to the process as described above in order to obtain a fermentate, and
ii) a step of mixing said fermentate from step i) with at least one pharmaceutically or cosmetically acceptable excipient.

It is noted that the invention is also characterized in that it produces metabolites which can be used in cosmetic or pharmaceutical composition.

Thus, the invention relates to a pharmaceutical or cosmetic composition obtained according to the process of the invention.

This pharmaceutical or cosmetic composition maybe in the form of an oil-in-water or water-in-oil emulsion, multiple emulsion, microemulsion, nanoemulsion, twin phase emulsion, PIT emulsion (phase inversion emulsion by temperature), stable dispersion of two immiscible phases by means of a gelling agent, stable dispersion of two immiscible phases by means of one or more surfactants, of a liquid, aqueous gel, fatty gel, hydroalcoholic gel, fatty phase, suspension, foaming or non-foaming solution, gel, lyophilized emulsion, powder, of liquid, foam, paste, ready-to-use drink, lotion, oil, gel, syrup, solid, mask, stick, tablet, capsule, spray or aerosol, gel capsule, jelly, creams, patch, shower gel, or shampoo. And that therefore the subject of the present invention also relates to a cosmetic or pharmaceutical composition characterized in that it contains the metabolites resulting from fermentation of bio-sourced ingredients according to the process of the invention for its use for the modulation of the epigenetic imprint and for modulating the enzymes regulating epigenetics.

The invention also relates to a cosmetic or pharmaceutical composition containing the metabolites resulting from fermentation of bio-sourced ingredients according to the process of the invention for its use in the treatment of age-related disorders and/or chronic inflammation.

The composition may enable the increase of telomere length and telomerase activity.

Alternatively or additionally, the composition modulates the epigenetic imprint by increasing DNA methylation, in particular the DNA of the promoter of the gene expressing interleukin-6 (of nucleotide sequence SEQ ID NO: 3), said interleukin-6 promoter having the nucleotide sequence SEQ ID NO: 1; and/or by increasing histone acetylation in the promoter region of the superoxide dismutase SOD2 gene, said superoxide dismutase SOD2 promoter having the nucleotide sequence SEQ ID NO: 2.

### Definitions

“Biosourced”: refers to a material or product manufactured with raw materials from biomass.

“Coculture”: concerns the joint culture of several organisms.

“Dispersion”: heterogeneous mixture of two finely mixed constituents without one phase being completely dissolved in the other.

“Enzyme: protein that accelerates chemical reactions in the body.

“Emulsion”: pharmaceutical preparation formed from two insoluble liquid phases, one of which is dispersed in the other in the form of globules.

“Epigenetics: corresponds to the study of changes in gene activity, not involving modification of the DNA sequence and which can be transmitted during cell divisions. Unlike mutations which affect the DNA sequence, epigenetic modifications are reversible.

“Fermentation”: Transformation that certain organic materials undergo under the action of enzymes secreted by microorganisms.

“Freeze-dried”: is said of a product presented in dehydrated form.

“Metabolite”: molecule resulting from the digestion of food by bacteria.

“Microbiota”: the set of microorganisms—bacteria, viruses, parasites and non-pathogenic fungi, known as commensals—which live in a specific environment of the host, such as the skin or the gastrointestinal system.

“Telomerase”: an enzyme present in eukaryotic organisms which, during DNA replication, has the mission of dressing chromosomes with telomeres, nucleotide sequences placed at their end and serving to preserve them.

“Surfactant: substance that modifies the surface tension between two surfaces.

### DETAILED DESCRIPTION OF THE INVENTION

For the clarity of the following explanations, we have decided to call “Spacebiome” a cosmetic or pharmaceutical composition containing metabolites produced by the fermentation process of bio-sourced ingredients at a temperature between 45 and 50.° C. for 180 hours with a co-culture of at least three bacterial strains that evolved under stress, through exposure to the space environment consisting in particular of exposure to UV radiation.

The subject of the present invention relates to a fermentation process intended to produce metabolites having properties of modulation of the epigenetic imprint. This process is characterized in that the fermentation is carried out with bio-sourced ingredients, at a temperature between 45 and 50° C., for 180 hours and by a co-culture of at least 3 bacterial strains evolved by exposure to the space environment, including exposure to UV radiation. The invention also relates to the composition obtained by the process, called “Spacebiome”.

The composition according to the invention, called “Spacebiome”, is characterized in that the bacteria used for the culture are taken from the following list: Lactobacillus fermentum, Lactobacillus casei, Lactobacillus acidophilus, Bifidobacterium longum, Bifidobacterium bifidum, Bifidobacterium adolescentis, Bifidobacterium infantis, Lactobacillus acidophilus, Lactobacillus brevis, Lactobacillus Jensenii, Lactobacillus paracasei subsp. Paracasei, Lactobacillus gasseri, Lactobacillus reuteri, Lactobacillus delbrueckii subsp. Bulgaricus, Lactobacillus helveticus, Lactococcus lactis, Lactobacillus casei subsp. Casei, Lactobacillus rhamnosus, Lactobacillus delbrueckii subsp. Delbrueckii, Enterococcus faecium and Streptococcus thermophilus, Akkermansia muciniphila. These bacteria are preferably isolated from plants or soil.

In one embodiment, the at least three strains are taken from the list consisting of Lactobacillus fermentum, Lactobacillus acidophilus, Lactobacillus brevis, Lactobacillus Jensenii, Lactobacillus paracasei subsp. Paracasei, Lactobacillus gasseri, Lactobacillus reuteri, Lactobacillus delbrueckii subsp. Bulgaricus, Lactobacillus helveticus, Lactococcus lactis, Lactobacillus casei subsp. Casei and Lactobacillus rhamnosus. In a particular embodiment, the at least three strains are taken from the list consisting of Lactobacillus fermentum, Lactobacillus casei and Lactobacillus acidophilus.

The above-mentioned bacterial strains are subjected to extreme stress. For this, the bacteria are exposed to space conditions consisting in particular of exposure to UV radiation. For this, a culture of bacteria, preferably frozen, is enclosed in an airtight container and sent into orbit for a minimum period of 1 month, in an environment with high UV radiation. These space conditions can be reproduced in the laboratory by exposing a culture of bacteria to high UV radiation. UV-B type at a dose of 0.1 to 3.0 W·m² for 1 week.

By exposing the bacteria to these conditions, the applicant noticed that the genome of the bacteria evolved and in particular that their epigenetic imprint evolved, which induced a modulation of gene expression, allowing them to withstand the extreme conditions to which they are subjected. The applicant noted in particular that there was a natural selection that took place to keep only the strongest strains and that these bacteria produced different enzymes and thus different metabolites than bacteria not exposed to these extreme conditions.

Advantageously, the biosourced ingredients used to obtain “Spacebiome” can be taken from the following list without this being limiting: olive leaves, fruits and bark, soybeans, propolis, pomegranate fruits and bark, fig fruits and bark, grapefruit, loquat, gingko biloba, algae, bean seeds, turmeric root, ginger root, flowers such as rose, apple fruits and bark. According to certain embodiments, biosourced ingredients are chosen from soybeans, olive leaves and a mixture of these.

Advantageously, the ingredients are of biosourced origin only, they are obtained without the use of pesticides and they are previously sterilized. Indeed, pesticides, in addition to their negative impact on biodiversity and human health, can inhibit fermentation. Sterilizing the ingredients before fermentation prevents contamination and the development of pathogenic microorganisms.

Advantageously, the composition according to the invention (“Spacebiome”) comprises metabolites which can be used in cosmetic or pharmaceutical compositions. The different types of compositions of the invention, called “Spacebiome”, as cited in the present invention were analyzed by HPLC chromatography. Analysis of the results demonstrated that the “Spacebiome” compositions formulated with different fermented ingredients all contain at least 150 metabolites in common.

The invention therefore also relates to a cosmetic or pharmaceutical composition for the application of “Spacebiome” containing the metabolites resulting from the fermentation of biosourced ingredients modulating the epigenetic imprint. The latter, through epigenetics, establish a direct relationship with the control of gene expression and can thus modulate it.

Unlike genetic modifications, epigenetic modifications, such as DNA methylation and histone modification are reversible and do not change the DNA sequence, but they can change the way the body reads and translates a DNA sequence. Epigenetic imprinting can be modified by environmental factors and can lead to altered gene expression and lead to problems such as: obesity, stress sensitivity, cardiovascular disease, premature aging. The metabolites of the “Spacebiome” composition modulate DNA methylation, i.e. the addition of a methyl group to DNA by modulating the enzymes histone acetyltransferase (HAT), histone deacetylase (HDAC) and histone methyltransferases (HMT), which are involved in modulating epigenetic imprinting. By modulating the genetic imprint, “Spacebiome” makes it possible to improve resistance to stress, reduce the level of circulating cortisol, improve sleep, improve long-term memory, improve liver health, improve fertility, improve the immune system, improve general health and particularly improve metabolism. According to a first aspect, the invention targets the cosmetic and non-therapeutic use of a composition according to the invention “Spacebiome” to delay and/or treat age-related manifestations in a healthy subject. According to a second aspect, the invention relates to a composition according to the invention, “Spacebiome”, in particular a pharmaceutical composition according to the invention, for its use in the treatment of age-related disorders and/or chronic inflammation. In some embodiments, the composition acts by modulating epigenetic imprinting, typically by modulating epigenetic regulating enzymes of the subject in need thereof.

Telomerase is an enzyme responsible for cell renewal, until our youth capital is exhausted. It is present in eukaryotic organisms and during DNA replication, its mission is to place telomeres on chromosomes, nucleotide sequences placed at their end and used to preserve them. As cell divisions progress, telomerase is no longer expressed in differentiated cells, telomeres disappear, chromosomes are damaged and the cell enters senescence. On the other hand, the enzyme is very active in germ lines. It therefore prevents stem cells from aging. It is considered that it could be one of the levers to activate to slow down or even stop aging. The metabolites of the “Spacebiome” composition increase telomerase activity, thus increasing the length of telomeres. Thus “Spacebiome” is therefore defined as a cosmetic or pharmaceutical composition capable of extending longevity and increasing telomere length and telomerase activity. Without wishing to be limited by any theory, the “Spacebiome” composition according to the invention modulates the epigenetic imprint by increasing DNA methylation, in particular the DNA of the promoter of the gene expressing interleukin-6 (of nucleotide sequence SEQ ID NO: 3), said interleukin-6 promoter having the nucleotide sequence SEQ ID NO: 1; and/or by increasing histone acetylation in the promoter region of the superoxide dismutase SOD2 gene, said superoxide dismutase SOD2 promoter having the nucleotide sequence SEQ ID NO: 2.

This cosmetic or pharmaceutical composition can be found in the form of an oil-in-water or water-in-oil emulsion, multiple emulsion, microemulsion, nanoemulsion, twin-phase emulsion, PIT emulsion, stable dispersion of two immiscible phases by means of a gelling agent, stable dispersion of two immiscible phases by means of one or more surfactants, of a liquid, aqueous gel, fatty gel, hydroalcoholic gel, fatty phase, suspension, foaming or non-foaming solution, gel, lyophilized emulsion or powder. It can also be presented in the form of liquids, foams, pastes, ready-to-use drinks, lotions, emulsions, oils, gels, syrups, solids, powders, masks, sticks, tablets, capsules, sprays, aerosols, capsules, jellies, syrups, creams, patches, shower gels, shampoos.

### EXAMPLES

The present invention will be better understood on reading the following examples which illustrate the invention in a non-limiting manner.

### Example 1: Evolution of Bacteria Subjected to Exposure to UV Rays

In order to analyze the effect of UV exposure on bacteria, a culture of Lactobacillus fermentum, Lactobacillus casei and Lactobacillus acidophilus were divided into two and one part was exposed to UV-B rays at a dose of 0.5 W·m². To measure the effect on epigenetic imprinting, the proportion of methylated motifs as well as the expression of 20 genes involved in metabolite production were measured. The results are presented in Table 1 below:

TABLE 1

| | Proportion of methylated |
|---|---|
| | motifs (%) |
| Lactobacillus fermentum (−UV-B) | 78.9 |
| Lactobacillus fermentum (+UV-B) | 98.2 |

By RT-PCR analysis, it was found that 15 of the 20 genes analyzed had a different expression level in the Lactobacillus fermentum strain subjected to UV-B.

The results show that exposure to UV-B rays modifies the epigenetic imprint as well as gene expression.

### Example 2: List of Metabolites Produced

Bacteria exposed to UV produce at least 150 metabolites which are: Glycine, alanine, serine, proline, baline, treonine, L-cysteine, isoleucine, leucine, aspartic acid, lysine, L-glutamic acid, L-methionine, histidine, phenylalanine, arginine, tyrosine, tryptophan, glutathione disulfide, glutathione (GHS), 4-hydroxy-L-proline, L-asparagine, L-glutamin, L-citrulline, β-alanine, Y-aminobutyric acid, betaine, L-homoserine, ornithine, choline, putrescine, spermidine, spermine, tyramine, D-glucose-1-phosphate, glucose-6-phosphate, fructose-6-phosphate, butyrate, zinc, cytosine, cytidine, uridine, adenosine, guanosine, cytidine 5′-phosphate, uridine phosphate, cyclic adenylic acid, 3′5-cyclic guanyl diphosphate, adenosine 5′-phosphate, guanosine pentaphosphate, uridine diphosphate, adenosine diphosphate, guanosine diphosphate, uracil, adenine, hypoxanthine, guanine, 3-methyl-2-oxobutanoic acid, fumaric acid, 2-oxoglutaric acid, cis-aconitic acid, glycolic acid, pyruvic acid, L-lactic acid, 3hydroxybutyric acid, 2hydroxybutyric acid, succinic acid, L-malic acid, citric acid, isocitric acid, D-gluconic Acid, 3-phospho-D-glyceric acid, glycerophosphoric acid, methylthioadenosine, uridine diphosphate glucose, xanthine, 7-methylguanine, thiamin, thiamine monophosphate, riboflavin, nicotinamide (niacinamin), nicotinic acid (niacin), pantothenic acid, pyridoxine, pyridoxamine, dihydrotachysterol, a-tocopherol, tocopherol acetate, biotin, trigonelline, lobeline, papaverine, imidazoleacetic acid, imidazolelactic acid, imazaquin, indole-3-carboxaldehyde, lumichrome, 5-Oxo2-tetrahydrofuran acid, chelidonic acid, gemfibrozil, diethyltoluamide, benzoic acid, 3-hydroxybenzoic acid, p-coumaric acid, terephthalic acid, phloretic acid, vanillic acid, cyanidin3-rutinoside, peonidin3-glucoside, daidzein, biochanin A, cupressuflavone, formononetin, 5,7-dimethoxyflavone, glycitein, puerarin, ononin, isoorientin (homoorientin), dalbergin, saikosaponin A, resveratrol, kaempferol, naringenin, eriodictyol, rutin, luteorin, apigenin-7-glucoside, glucoluteolin, myricitrin, liquiritigenin, acacetin, prunin (naringenin7-OBD-glucoside), baicalin, saponarin, neoeriocitrin, vitexin (apigenin-8-glucoside), chrysoeriol, Datiscentin, 5-methoxyindoleacetic acid, butyric acid, isovaleric acid (methylbutyric acid), glutaric acid, pelargonic acid, alpha lipoic acid, malronic acid, glucaric acid, abietic acid, trans-aconitic acid, and D-glyceric acid, 2-hydroxyvaleric acid.

In order to study the effect of UV exposure on the production of metabolites, a culture of Lactobacillus fermentum, Lactobacillus casei and Lactobacillus acidophilus were divided into two and one part was exposed to UV-B rays at a dose of 0.5 W·m². Both cultures were placed in the presence of soy milk which was fermented for 3 days at 37° C. The amount of metabolites was measured by HPLC.

It was noted that UV exposure significantly increased the production of the metabolites mentioned above.

### Example 3: Modulation of Epigenetic Imprinting

A study was conducted to evaluate the effect of ingestion of “Spacebiome”, a pharmaceutical composition comprising metabolites produced by the fermentation of soybeans (Glycine max) by UV-B evolved bacteria at a dose of 0.5 W/m2 Lactobacillus fermentum, Lactobacillus casei, Lactobacillus acidophilus, Bifidobacterium longum on epigenetic imprinting. For this, 60 male and female participants were recruited. The 60 subjects were randomly divided into 3 groups of 20 people. For 3 months, the first group (“Spacebiome”) ingested a daily dose of 500 mg of “Spacebiome”, the second group (“Placebo”) ingested a placebo, the third group (“Metabolites”) ingested a daily dose of 500 mg of a composition containing metabolites from the same fermentation as “Spacebiome” but without the bacteria used having been exposed to UV. A genome-wide DNA methylation analysis was performed on saliva samples using the Illumina Methylation Epic Array and biological age (DNAmAge) was estimated. The results are presented in Table 2 below:

TABLE 2

| | DNAmage (Years) |
|---|---|
| Placebo | +0.20 ± 0.48 |
| Metabolites | −0.42 ± 0.39 |
| Spacebiome | −1.91 ± 0.31 |

It is observed that biological age decreased at the end of the study in the “Spacebiome” and Metabolites group, while it increased in the placebo group. The results of the “Spacebiome” group are better than the results of the Metabolites group. These results show that “Spacebiome” is effective in modulating the epigenetic imprint.

### Example 4: Modulation of Epigenetic Enzymes

A study was conducted to investigate the effect of “Spacebiome”, a pharmaceutical composition comprising metabolites produced by the fermentation of olive leaves (Olea europaea) by bacteria evolved by exposure to space conditions for 6 months Lactobacillus fermentum, Lactobacillus acidophilus, Lactobacillus brevis, Lactobacillus Jensenii, Lactobacillus paracasei subsp. Paracasei, Lactobacillus gasseri, Lactobacillus reuteri, Lactobacillus delbrueckii subsp. Bulgaricus, Lactobacillus helveticus, Lactococcus lactis, Lactobacillus casei subsp. Casei and Lactobacillus rhamnosus. on the gene expression of enzymes involved in the modulation of epigenetic imprinting. For this, HeLa cells were incubated with “Spacebiome” for 48 hours. The amount of histone acetyltransferase (HAT), histone deacetylase (HDAC) and histone methyltransferases (HMT) enzymes were measured by assessing their activity.

The results are presented in Table 3 below:

TABLE 3

| | 0 | 10⁻⁹ | 10⁻⁸ | 10⁻⁷ |
|---|---|---|---|---|
| Activity HAT (%) | 100 ± 4.2 | 82.6 ± 2.1 | 63.1 ± 2.9 | 21.2 ± 1.7 |
| Activity HDAC (%) | 100 ± 3.6 | 89.3 ± 3.5 | 78.4 ± 2.8 | 71.7 ± 2.4 |
| Activity HMT (%) | 100 ± 4.5 | 95.5 ± 4.3 | 91.2 ± 4.5 | 89.6 ± 2.9 |

We observed that incubation with “Spacebiome” increases epigenetic enzymes and that this increase is dose-dependent. These results show that “Spacebiome” modulates enzymes regulating epigenetics.

### Example 5: Increased Telomerase

A study was conducted to study the effect of “Spacebiome” on telomeres. For this, 60 male and female participants were recruited and divided into 3 groups of 20 participants. For 3 months, the first group (“Spacebiome”) ingested a daily dose of 500 mg of “Spacebiome”, a pharmaceutical composition comprising metabolites produced by the fermentation of soybeans (Glycine max) by bacteria evolved by exposure to space conditions for 6 months Lactobacillus fermentum, Lactobacillus casei, Lactobacillus acidophilus, Bifidobacterium longum. The second group (“Placebo”) ingested a placebo, the third group (“Metabolites”) ingested a daily dose of 500 mg of a composition containing metabolites from the same fermentation as “Spacebiome” but without the bacteria used having been exposed to UV. Telomere length of leukocytes isolated from blood was measured before and after the study period.

The results are presented in Table 4 below:

TABLE 3

| | Placebo | | Metabolites | | Spacebiome | |
|---|---|---|---|---|---|---|
| | before | after | before | after | before | after |
| Telomere | 6271 ± | 6350 ± | 6209 ± | 6687 ± | 6187 ± | 7746 ± |
| length (bases) | 590 | 620 | 535 | 471 | 521 | 438 |

| | Placebo | Metabolites | Spacebiome |
|---|---|---|---|
| Change in | +0.2 ± 0.4 | +5.6 ± 1.2 | +21.7 ± 1.9 |
| telomerase activity (%) | | | |

It was noted that telomere length increased in the Metabolites and “Spacebiome” group, while placebo consumption had no significant effect. It was also noted that telomerase activity increased in the Metabolites and Spacebiome group. These results demonstrate that “Spacebiome” increases telomere length and increases telomerase activity.

### Example 6: Food Supplement

| | |
|---|---|
| Spacebiome | 50-100 mg |
| Dextrin | 300-450 mg |
| Maltose | 300-500 mg |
| Fruit juice | 50-90 mg |
| Citric acid | 100-200 mg |
| Natural flavors | 10-20 mg |

This formula is a food supplement to be taken morning and evening. It has epigenetic modulating properties. Users have noticed weight loss after 3 months of regular consumption.

### Example 7: Anti-Wrinkle O/W Gel Emulsion

| | | |
|---|---|---|
| Spacebiome | 2 | g |
| Carbopol 981 | 0.6 | g |
| Ethyl alcohol | 15 | g |
| Volatile silicone oil | 3 | g |
| Purcellin oil | 3 | g |
| Scent | 0.4 | g |
| Triethanolamine | 0.2 | g |
| Preservatives | 0.3 | g |
| Demineralized water | 100 | g |

This gel emulsion is to be applied once a day. Users have noticed a visible anti-wrinkle effect on the skin by local application such as on dark circles on the face.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. Fermentation process, intended to produce metabolites having properties of modulation of the epigenetic imprint, characterized in that the fermentation is carried out at a temperature between 45 and 50° C. for 180 hours by mixing bio-sourced ingredients with a co-culture of at least 3 bacterial strains each presenting an epigenetic imprint modified by exposure to conditions of a space environment consisting in particular of exposure to UV radiation.

2. Fermentation process according to claim 1, characterized in that the bacteria used for the fermentation are taken from the following list: Lactobacillus fermentum, Lactobacillus casei, Lactobacillus acidophilus, Bifidobacterium longum, Bifidobacterium bifidum, Bifidobacterium adolescentis, Bifidobacterium infantis, Lactobacillus acidophilus, Lactobacillus brevis, Lactobacillus Jensenii, Lactobacillus paracasei subsp. Paracasei, Lactobacillus gasseri, Lactobacillus reuteri, Lactobacillus delbrueckii subsp. Bulgaricus, Lactobacillus helveticus, Lactococcus lactis, Lactobacillus casei subsp. Casei, Lactobacillus rhamnosus, Lactobacillus delbrueckii subsp. Delbrueckii, Enterococcus faecium, Streptococcus thermophilus and Akkermansia muciphila.

3. Fermentation process according to claim 1 or claim 2, characterized in that the conditions of the spatial environment consist of exposure to UV radiation of the UV-B type at a dose of 0.1 to 3.0 W·m2.

4. Fermentation process according to claims 1 to 3, characterized in that the fermentation is carried out with bio-sourced ingredients only, which are obtained without the use of pesticides, and which are previously sterilized.

5. Fermentation process according to claims 1 to 4, characterized in that the bio-sourced ingredients are chosen from soybeans, olive leaves and a mixture of these.

6. Process for the preparation of a pharmaceutical or cosmetic composition comprising:
a fermentation step according to the process as described in any one of claims 1 to 5 in order to obtain a fermentate, and
a step of mixing said fermentate from step i) with at least one pharmaceutically or cosmetically acceptable excipient.

7. Pharmaceutical or cosmetic composition obtained according to the process of claim 6, characterized in that said pharmaceutical or cosmetic composition is in the form of an oil-in-water or water-in-oil emulsion, multiple emulsion, microemulsion, nanoemulsion, twin-phase emulsion, PIT emulsion, stable dispersion of two immiscible phases by means of a gelling agent, stable dispersion of two immiscible phases by means of one or more surfactants, of a liquid, aqueous gel, fatty gel, hydroalcoholic gel, fatty phase, suspension, foaming or non-foaming solution, gel, lyophilized emulsion, powder, of liquid, foam, paste, ready-to-use drink, lotion, oil, gel, syrup, solid, mask, stick, tablet, capsule, spray or aerosol, gel cap, jelly, cream, patch, shower gel, or shampoo.

8. Pharmaceutical composition according to claim 7 for its use as medicine.

9. Pharmaceutical composition according to claim 8 for its use in the treatment of chronic inflammation.

10. A method of producing metabolites having properties of a modulated epigenetic imprint, comprising:
modifying the epigenetic imprint of a co-culture of at least three bacterial strains by exposing said co-culture to conditions of UV space radiation,
mixing said co-culture with bio-sourced ingredients to produce a mixture, and
fermenting said mixture at a temperature between 45 and 50° C. for 180 hours to produce a fermentate.

11. The method of claim 10 wherein said co-culture is selected from three or more of the group consisting of Lactobacillus fermentum, Lactobacillus casei, Lactobacillus acidophilus, Bifidobacterium longum, Bifidobacterium bifidum, Bifidobacterium adolescentis, Bifidobacterium infantis, Lactobacillus acidophilus, Lactobacillus brevis, Lactobacillus Jensenii, Lactobacillus paracasei subsp. Paracasei, Lactobacillus gasseri, Lactobacillus reuteri, Lactobacillus delbrueckii subsp. Bulgaricus, Lactobacillus helveticus, Lactococcus lactis, Lactobacillus casei subsp. Casei, Lactobacillus rhamnosus, Lactobacillus delbrueckii subsp. Delbrueckii, Enterococcus faecium, Streptococcus thermophilus and Akkermansia muciphila.

12. The method of claim 10 wherein said step of modifying said co-culture comprises exposing said co-culture to UV radiation of the UV-B type at a dose of 0.1 to 3.0 W/m2.

13. The method of claim 12 wherein said bio-sourced ingredients are selected from one or more of the group consisting of soybeans and olive leaves.

14. The method of claim 13 wherein said bio-sourced ingredients are sterilized prior to the mixing step.

15. The method of claim 10 further comprising the step of mixing said fermentate with a pharmaceutical excipient.

16. The method of claim 10 further comprising the step of mixing said fermentate with a cosmetic excipient.
